(19) United States
(12) Patent Application Publication
(10) Pub. No.: US 2020/0263254 A1
(43) Pub. Date: Aug. 20, 2020

(54) METHOD FOR DETERMINING THE RESPONSE OF A MALIGNANT DISEASE TO AN IMMUNOTHERAPY

(71) Applicant: Dimo Dietrich, Berlin (DE)

(72) Inventor: Dimo Dietrich, Berlin (DE)

(21) Appl. No.: 15/733,039

(22) PCT Filed: Nov. 4, 2018
(86) PCT No.: PCT/EP2018/080073

§ 371 Date: Apr. 24, 2020

(30) Foreign Application Priority Data
Nov. 5, 2017 (DE) 10 2017 125 780.2

(51) Int. Cl. C12Q 1/6886; A61K 39/395; A61P 35/00

(52) U.S. Cl. CPC C12Q 1/6886; A61K 39/3955; C12Q 2600/106; C12Q 2600/154; A61P 35/00

(57) ABSTRACT

A method is useful for predicting a response of a malignant disease to an immunotherapy which inhibits the PD-1 immune checkpoint signaling pathway. The method is characterized in that at least a part of an immunoregulatory gene selected from CTLA4, CD86, CD28, CD80 and/or ICOS of cells of the malignant disease and/or of immune cells interacting with cells of the malignant disease is subjected to a DNA methylation analysis, and the result is used to predict the response of the malignant disease to the immunotherapy.

### CROSS-REFERENCE TO RELATED APPLICATIONS

The present application is a National stage entry under § 371 of International Application No. PCT/EP2018/080073, filed on Nov. 4, 2018, and which claims the benefit of German Application No. 10 2017 125 780.2, filed on Nov. 5, 2017, the contents of each of which are hereby incorporated by reference in their entireties.

### REFERENCE TO A SEQUENCE LISTING

The present application is accompanied by an ASCII text file which has been submitted via EFS-Web as a computer readable form containing the sequence listing, titled “2020-04-17-PCTUS_Sequence-Listing_revised_ST25.txt”, created on Apr. 17, 2020, with the file size of 991,691 bytes, which is incorporated by reference in its entirety.

### BACKGROUND OF THE INVENTION

### Field of the Invention

The invention relates, inter alia, to predictive molecular diagnostic methods and predictive biomarkers and their uses in oncological medicine for predicting the response of a malignant disease to immunotherapy. Furthermore, the invention relates to a kit for carrying out the specified methods.

### Discussion of the Background

Personalized medicine is based on the development of therapies that are tailored to patients with specific diseases. Immunotherapeutic treatments with so-called immune checkpoint inhibitors represent a promising approach in oncology, because they can also show exceptional results in advanced tumor diseases. A problem is, however, that even patients with identical clinical symptoms can respond differently to the same therapy or the same therapeutic drug.

Predictive molecular diagnostics (“companion diagnostics” and “complementary diagnostics”) and predictive biomarkers (“companion biomarkers” and “complementary biomarkers”) can help predict how well a patient will respond to a particular treatment or drug. For example, it is known from DE 10 2016 005 947 B3 that a DNA methylation analysis of the PD-1 gene of cells of a malignant disease or of T lymphocytes interacting with cells of the malignant disease enables a prediction of whether the patient will respond to immunotherapy with pharmaceutical compounds that inhibit the PD-1 receptor or its ligand.

In order to further improve the benefit-risk ratio of such immunotherapies, it is necessary to support clinical decision making by more precise subtyping of patient groups. There is a need for additional or complementary predictive methods and biomarkers to guide patient selection more reliably in view of a wide range of emerging treatment options.

### SUMMARY OF THE INVENTION

Against this background, a first aspect of the present invention provides a method for predicting a response of a malignant disease to an immunotherapy designed to inhibit a PD-1 immune checkpoint signaling pathway. The method is characterized in that at least a part of an immunoregulatory gene selected from CTLA4, CD86, CD28, CD80 and/or ICOS of cells of said malignant disease and/or of immune cells interacting with cells of said malignant disease is subjected to DNA methylation analysis. Subsequently, based on the result of the DNA methylation analysis, i.e. based on the presence, absence and/or level of DNA methylation of said immunoregulatory gene, the response of the malignant disease to said immunotherapy is predicted.

The second aspect of the invention provides a method for selecting a patient suffering from a malignant disease for an immunotherapy designed to inhibit a PD-1 immune checkpoint signaling pathway. The method comprises the steps of A) providing cells of said malignant disease and/or immune cells interacting with cells of said malignant disease from the patient, B) performing a DNA methylation analysis of at least a part of an immunoregulatory gene selected from CTLA4, CD86, CD28, CD80 and/or ICOS from the cells of the malignant disease and/or from the immune cells interacting with the cells of the malignant disease from A), and C) selecting the patient for said immunotherapy on the basis of the presence, absence and/or level of DNA methylation of said immunoregulatory gene examined or determined in B).

The third aspect of the invention provides a use of DNA methylation analysis of at least a part of an immunoregulatory gene selected from CTLA4, CD86, CD28, CD80 and/or ICOS of cells of a malignant disease and/or of immune cells interacting with cells of the malignant disease for predicting a response of the malignant disease to an immunotherapy, for the individualized selection of an immunotherapy for the malignant disease and/or for selecting a patient suffering from the malignant disease for an immunotherapy, wherein the immunotherapy is designed to inhibit a PD-1 immune checkpoint signaling pathway.

The fourth aspect of the invention provides a use of the presence, absence or level of DNA methylation of at least a part of an immunoregulatory gene selected from CTLA4, CD86, CD28, CD80 and/or ICOS of cells of a malignant disease and/or of immune cells interacting with cells of the malignant disease as a biomarker for predicting a response of said malignant disease to immunotherapy, for the individualized selection of an immunotherapy for said malignant disease and/or for selecting a patient suffering from said malignant disease for an immunotherapy, wherein said immunotherapy is designed to inhibit a PD-1 immune checkpoint signaling pathway.

According to a fifth aspect, the invention provides a kit for carrying out one of the aforementioned methods or for one of the aforementioned uses. The kit comprises reagents for DNA methylation analysis of at least part of an immunoregulatory gene selected from CTLA4, CD86, CD28, CD80 and/or ICOS of cells of a malignant disease and/or of immune cells interacting with cells of the malignant disease in order to determine the presence, absence and/or level of DNA methylation of said immunoregulatory gene.

### Definitions and General Explanations

In this description, various documents are cited to provide a general technical background with respect to the present invention. The disclosure and teaching of these documents are hereby incorporated by reference in its entirety to supplement the following description in order to avoid repetition.

The following definitions and general explanations are intended to guide and assist the skilled reader in understanding, interpreting and practicing the present invention. Unless otherwise stated, all technical and scientific terms shall have the meaning which corresponds to the usual understanding of a person of ordinary skill in the art in the field of the present invention.

The different aspects and variants of the present invention involve techniques and methods that are routine practice in molecular biology. In particular, DNA methylation analysis for determination of the methylation of a CpG dinucleotide is part of the technical knowledge of a molecular biologist or geneticist. Useful laboratory manuals for these techniques and methods are readily available to the skilled person, such as “Molecular Cloning. A Laboratory Manual” by M. R. Green and J. Sambrook, 4th Ed., 2012, Cold Spring Harbor Laboratory Press.

As they are used herein, indefinite articles such as “a” or “one” include the possibility that two or more of these features may also be present.

As used herein, the term “malignant disease” or “malignant diseases” includes such clinical conditions that are characterized by a course of disease that is progressively destructive and may eventually lead to the death of the patient. Malignant diseases comprise malignant tissue formations, for example neoplasias or tumors, wherein the malignancy can be characterized by uncontrolled, space-consuming, displacing, infiltrative and/or invasive growth. Malignant tumors are generally capable of forming metastases. Malignant tumors include carcinomas, sarcomas, melanomas, glioblastomas, blastomas, seminomas and teratomas. Malignant diseases also include hematological malignant diseases, i. e. malignant diseases that affect the blood or hematopoietic system, such as leukemias, lymphomas, myeloproliferative diseases and myelodysplastic syndromes. Leukemias comprise a group of malignant diseases in which immature hematopoietic cells have undergone malignant changes, proliferate excessively and lead to an accumulation of cells in the peripheral blood. Lymphomas comprise diseases in which cells of the lymphatic system are degenerated. Myeloproliferative diseases comprise a group of diseases in which one or more hematopoietic cell lines are highly proliferated. Myelodysplastic syndromes comprise a clonal expansion of precursor cells of all hematopoietic cell lines, based on a chronic differentiation disorder of the hematopoietic stem cells.

“Immunotherapy” or “immunotherapeutic treatment” is used herein as a collective term for all methods of treatment aimed at influencing the activity of the immune system. Immunotherapy can be directed at strengthening or weakening the effect of the immune system. In certain variants of the invention, an immunotherapy comprises treatment with pharmaceutical compounds to strengthen the organism's own immune response against the malignant disease. Such pharmaceutical compounds include immune checkpoint inhibitors such as monoclonal antibodies which specifically bind to immune checkpoints and thus prevent their (in particular anti-inflammatory) signaling. Another possibility are compounds that inhibit the expression of immune checkpoints, for example by RNA interference.

The “response” to immunotherapy can be assessed according to the immune-related Response Evaluation Criteria In Solid Tumors-irRECIST (Nishino et al., J Immunother Cancer, 2014, 2:17, Wolchok et al., Clin Cancer Res. 2009, 15:7412-20). In particular, a response to immunotherapy may be characterized by complete or partial remission, by an unchanged (stable) state, and by a delay in one or more of the following events: death, recurrence, occurrence of lymph node metastases, occurrence of distant metastases, progression of the malignant disease. A response to immunotherapy may also be characterized by a delayed increase or decrease in another parameter specific to the malignant disease. For example, a decrease or delayed increase in blood levels of prostate-specific antigen (PSA) may indicate a prostate cancer response to immunotherapy. Failure to respond may be characterized by an increasing or accelerated increase in the extent of the malignant disease. The degree of the malignant disease before the beginning of the therapy can serve as a comparative measure. The extent of the disease can be characterized by the number of malignant cells, the number of metastases or the size of the malignant tumor.

As used herein, “inhibiting a PD-1 immune checkpoint pathway” or “inhibiting a CTLA4 immune checkpoint pathway” means to slow down, inhibit or prevent one or more reactions of a chemical, biological and/or physical nature mediated by an interaction of the PD-1 immune checkpoint with its ligand PD-L1 and/or PD-L2, or of the CTLA4 immune checkpoint with its ligand CD86 and/or CD80, respectively.

“DNA methylation” is considered to be the biochemical or chemical coupling of methyl groups to specific nucleotides of DNA. In the context of the present invention, “DNA methylation” refers to the presence of a methyl group on the fifth carbon atom of a cytosine (5-methylcytosine) located in a CpG dinucleotide context. A “CpG dinucleotide” is a DNA motif which has the nucleoside sequence cytidine-phosphate-guanosine in the generally recognized reading direction from 5′ to 3′. Guanosine consists of the nucleobase guanine and the sugar β-D-ribose. Cytidine consists of the nucleobase cytosine and the sugar β-D-ribose.

A “DNA methylation analysis” in the sense of the present invention therefore comprises the determination of such DNA methylation of one or more CpG dinucleotides from a specific sequence context. In various variants of the present invention, “DNA methylation analysis” shall be understood as the determination of whether the cytosine is present in said CpG dinucleotide(s) in methylated form. If a large number of genome copies are examined, DNA methylation analysis can also be used to determine the level of DNA methylation. For this purpose, e. g. an average presence of the methylated form of cytosine in the CpG dinucleotide(s) contained in the large number of genome copies is determined, hereinafter also referred to as “degree of methylation”.

“Co-methylation” means a correlation of DNA methylation between two or more CpG dinucleotides. Such correlating methylations regularly occur in CpG dinucleotides that are adjacent in the genome and/or that are located within adjacent, structurally and/or functionally related genes that can be expressed together. DNA methylation of one gene can therefore be used to draw conclusions about DNA methylation of the other, co-methylated gene.

A “gene” in the sense of the present invention is a section of DNA which comprises regulatory, transcribed and/or functional sequence regions and thus contains the basic information for the production of a biologically active RNA. In particular, a gene can also comprise those elements, such as promoter, transcription factor binding sites, CpG islands, open chromatin, enhancer and silencer. CTCF binding sites, which fulfil a regulatory function in the transcription of the gene.

The nomenclature for the designation of genes and their nucleotides is based on the recommendation of the “Human Genome Organisation Gene Nomenclature Committee” (HGNC) as of 31 Oct. 2017; for example, a gene stem is designated by italic Latin capital letters (e.g. (CTLA4, CD86). The genes described herein are publicly available via the “GenBank” of the National Institute of Health, USA, as of 31 Oct. 2017 (Benson D. A. et al., Nucleic Acids Research, 2013, 41. D36-42).

When reference is made in the following description to specific DNA sequences (SEQ ID NOs), this shall always include sequence variants with at least 95%, at least 96%, at least 97%, at least 98% or at least 99% sequence identity with the said DNA sequence. The sequence identity of two nucleic acid sequences can be determined, for example, with the ClustalW algorithm (Thompson et al., Nucleic Acids Research, 1994, 22, 4673-4680).

For the purposes of the present invention, “immune cells interacting with cells of the malignant disease” comprises those immune cells which are in specific contact or can be in specific contact with cells of the malignant disease, for example, via a ligand-receptor bond. The ligand can be located on the surface of malignant disease cells, for example an MHC peptide complex, an MHC 1 antigen complex or an epitope. The receptor can be located on the surface of the immune cells, for example a T-cell receptor or B-cell receptor. Alternatively, the ligand can be located on the surface of the immune cells and the receptor can be located on the surface of the cells of the malignant disease. “Immune cells interacting with the cells of the malignant disease” therefore also includes, for example. T lymphocytes and/or B lymphocytes which, through contact with an antigen or antigen-presenting cells, have been enabled (activated) to interact specifically with cells of the malignant disease via one of the aforementioned ligand-receptor bonds without themselves having previously come into contact with the corresponding cells of the malignant disease. An antigen-presenting cell can be a dendritic cell, a macrophage or a B lymphocyte, for example. Activation occurs, for example, via ligand-receptor binding between T lymphocytes and an antigen-presenting cell, which presents an antigen derived from a cell of the malignant disease. The ligand, for example an MHC II antigen complex, is located on the surface of the antigen-presenting cells and the T-cell receptor is located on the surface of the T lymphocyte. Another form of interaction between T lymphocytes and cells of the malignant disease in the sense of the present invention involves adenosine produced by the cells of the malignant disease being bound by a receptor on the surface of the T lymphocytes. It is also possible that the adenosine is produced by T lymphocytes and the receptor is located on the surface of cells of the malignant disease. In the sense of the present invention. “immune cells interacting with cells of the malignant disease” further includes such immune cells, in particular T lymphocytes and/or B lymphocytes, which interact with the cells of the malignant disease via growth factors and/or cytokines.

“Biomarkers” are characteristic indicators and/or biological features that can be objectively measured and that allow conclusions to be drawn about the status of a normal biological process or a pathological process in an organism, or the response of a normal or pathological process to an intervention, such as surgery, radiation or pharmaceutical treatment. Biomarkers are often (bio)chemical substances such as proteins, hormones, metabolites, sugars and nucleic acids, as well as modifications thereof.

Both the above general description and the following detailed description should be understood as examples and are intended to explain the claimed invention. Further advantages and features of the invention are apparent from the following description, drawings and claims. While the invention is described on the basis of preferred embodiments, many further variations can be made without departing from the scope of the present invention. Therefore, it is intended that the claims cover variations and combinations of features that are included in the actual scope of the invention, even if they are not expressly mentioned in the claims.

### BRIEF DESCRIPTION OF FIGURES

FIG. 1 shows a box plot of the distribution of CTLA4 DNA methylation in malignant melanomas of patients with progressive disease, stable disease, partial and complete remission during immunotherapy designed to inhibit the PD-1 immune checkpoint signaling pathway. DNA methylation was determined before starting immunotherapy. Patients were grouped retrospectively according to irRECIST criteria.

FIG. 2 shows a Kaplan-Meier analysis of the overall survival of 50 patients with malignant melanomas during immunotherapy designed to inhibit the PD-1 immune checkpoint signaling pathway. The patients were grouped in a three-level classification based on CTLA4 DNA methylation. The lower tertile comprises the 17 patients with the lowest measured DNA methylation, the upper tertile comprises the 17 patients with the highest measured DNA methylation and the middle tertile comprises the remaining 16 patients.

FIG. 3 shows a co-methylation matrix of different gene loci of CTLA4. CD28. CD80. CD86 and ICOS in 419 urothelial carcinomas of the bladder.

FIG. 4 shows a co-methylation matrix of different gene loci of CTLA4, CD28, CD80, CD86 and ICOS in 797 breast cancer tumors.

FIG. 5 shows a co-methylation matrix of different gene loci of CTLA4, CD28, CD80. CD86 and ICOS in 530 squamous cell carcinomas of the head and neck.

FIG. 6 shows a co-methylation matrix of different gene loci of CTLA4, CD28, CD80, CD86 and ICOS in 325 clear cell renal cell carcinomas.

FIG. 7 shows a co-methylation matrix of different gene loci of CTLA4, CD28, CD80, CD86 and ICOS in 475 adenocarcinomas of the lung.

FIG. 8 shows a co-methylation matrix of different gene loci of CTLA4, CD28, CD80, CD86 and ICOS in 370 squamous cell carcinomas of the lung.

FIG. 9 shows a co-methylation matrix of different gene loci of CTLA4, CD28, CD80, CD86 and ICOS in 265 sarcomas.

FIG. 10 shows a co-methylation matrix of different gene loci of CTLA4, CD28, CD80, CD86 and ICOS in 473 cutaneous melanomas.

### BRIEF DESCRIPTION OF THE SEQUENCES

SEQ ID NO:1: CD28, CTLA4, ICOS gene locus 2:203551590-204126647; SEQ ID NO:2: CD80 gene locus 3:119523584-119573836; SEQ ID NO:3: CD86 gene locus 3:122039741-122154807; SEQ ID NO:4: ACTB qPCR reverse primer; SEQ ID NO:5: ACTB qPCR forward primer; SEQ ID NO:6: ACTB qPCR detection probe; SEQ ID NO:7: ACTB qPCR amplicon; SEQ ID NO:8: genomic sequence of ACTB qPCR amplicon; SEQ ID NO:9: CTLA4 qPCR forward primer; SEQ ID NO: 10: CTLA4 qPCR reverse primer: SEQ ID NO: 11: CTLA4 qPCR detection probe; SEQ ID NO:12: CTLA4 qPCR amplicon (methylated); SEQ ID NO: 13: genomic sequence of CTLA4 qPCR amplicon; SEQ ID NO: 14: CTLA4 gene locus 2:203867786-203873960; SEQ ID NO: 15: CTLA4 promoter 2:203861999-203868480; SEQ ID NO: 16: target sequence of probe cg15765889 from the Infinium HumanMethylation450 BeadChip; SEQ ID NO: 17: target sequence of probe cg14308944 from the Infinium HumanMethylation450 BeadChip: SEQ ID NO: 18: target sequence of probe cg11028872 from the Infinium HumanMethylation450 BeadChip; SEQ ID NO: 19: target sequence of probe cg15022671 from the Infinium HumanMethylation450 BeadChip; SEQ ID NO:20: target sequence of probe cg21078108 from the Infinium HumanMethylation450 BeadChip: SEQ ID NO:21: target sequence of probe cg27614178 from the Infinium HumanMethylation450 BeadChip; SEQ ID NO:22: target sequence of probe cg10240150 from the Infinium HumanMethylation450 BeadChip: SEQ ID NO:23: target sequence of probe cg13651908 from the Infinium HumanMethylation450 BeadChip; SEQ ID NO:24: target sequence of probe cg13790288 from the Infinium HumanMethylation450 BeadChip; SEQ ID NO: 25: target sequence of probe cg21911000 from the Infinium HumanMethylation450 BeadChip: SEQ ID NO:26: target sequence of probe cg22309950 from the Infinium HumanMethylation450 BeadChip; SEQ ID NO:27: target sequence of probe cg01206398 from the Infinium HumanMethylation450 BeadChip; SEQ ID NO:28: target sequence of probe cg09345839 from the Infinium HumanMethylation450 BeadChip; SEQ ID NO:29: target sequence of probe cg12005412 from the Infinium HumanMethylation450 BeadChip: SEQ ID NO:30: target sequence of probe cg05074138 from the Infinium HumanMethylation450 BeadChip; SEQ ID NO:31: target sequence of probe cg14288266 from the Infinium HumanMethylation450 BeadChip; SEQ ID NO:32: target sequence of probe cg05092371 from the Infinium HumanMethylation450 BeadChip; SEQ ID NO:33: target sequence of probe cg24077172 from the Infinium HumanMethylation450 BeadChip; SEQ ID NO:34: target sequence of probe cg00372692 from the Infinium HumanMethylation450 BeadChip; SEQ ID NO:35: target sequence of probe cg15247069 from the Infinium HumanMethylation450 BeadChip; SEQ ID NO:36: target sequence of probe cg18561976 from the Infinium HumanMethylation450 BeadChip: SEQ ID NO:37: target sequence of probe cg110114877 from the Infinium HumanMethylation450 BeadChip; SEQ ID NO:38: target sequence of probe cg13756014 from the Infinium HumanMethylation450 BeadChip: SEQ ID NO:39: target sequence of probe cg08185695 from the Infinium HumanMethylation450 BeadChip; SEQ ID NO:40: target sequence of probe cg26836175 from the Infinium HumanMethylation450 BeadChip; SEQ ID NO:41: target sequence of probe cg20149531 from the Infinium HumanMethylation450 BeadChip: SEQ ID NO:42: target sequence of probe cg25683810 from the Infinium HumanMethylation450 BeadChip; SEQ ID NO:43: target sequence of probe cg09861034 from the Infinium HumanMethylation450 BeadChip: SEQ ID NO:44: target sequence of probe cg04098585 from the Infinium HumanMethylation450 BeadChip; SEQ ID NO:45: target sequence of probe cg07930752 from the Infinium HumanMethylation450 BeadChip; SEQ ID NO:46: target sequence of probe cg24336674 from the Infinium HumanMethylation450 BeadChip: SEQ ID NO:47: target sequence of probe cg02099418 from the Infinium HumanMethylation450 BeadChip; SEQ ID NO:48: target sequence of probe cg23012700 from the Infinium HumanMethylation450 BeadChip: SEQ ID NO:49: target sequence of probe cg26091609 from the Infinium HumanMethylation450 BeadChip; SEQ ID NO:50: target sequence of probe cg22572158 from the Infinium HumanMethylation450 BeadChip; SEQ ID NO:51: target sequence of probe cg08460026 from the Infinium HumanMethylation450 BeadChip: SEQ ID NO:52: target sequence of probe cg18914852 from the Infinium HumanMethylation450 BeadChip; SEQ ID NO:53: target sequence of probe cg18219180 from the Infinium HumanMethylation450 BeadChip; SEQ ID NO:54: target sequence of probe cg21423458 from the Infinium HumanMethylation450 BeadChip; SEQ ID NO:55: target sequence of probe cg17751550 from the Infinium HumanMethylation450 BeadChip: SEQ ID NO:56: target sequence of probe cg15344028 from the Infinium HumanMethylation450 BeadChip; SEQ ID NO:57: target sequence of probe cg21139795 from the Infinium HumanMethylation450 BeadChip; SEQ ID NO:58: target sequence of probe cg12978275 from the Infinium HumanMethylation450 BeadChip: SEQ ID NO:59: target sequence of probe cg02470871 from the Infinium HumanMethylation450 BeadChip; SEQ ID NO:60: target sequence of probe cg21572897 from the Infinium HumanMethylation450 BeadChip: SEQ ID NO:61: target sequence of probe cg06045968 from the Infinium HumanMethylation450 BeadChip; SEQ ID NO:62: target sequence of probe cg13458803 from the Infinium HumanMethylation450 BeadChip; SEQ ID NO:63: target sequence of probe cg06300880 from the Infinium HumanMethylation450 BeadChip; SEQ ID NO:64: target sequence of probe cg13913728 from the Infinium HumanMethylation450 BeadChip; SEQ ID NO:65: target sequence of probe cg11874272 from the Infinium HumanMethylation450 BeadChip; SEQ ID NO: 66: target sequence of probe cg04387658 from the Infinium HumanMethylation450 BeadChip; SEQ ID NO: 67: target sequence of probe cg01878435 from the Infinium HumanMethylation450 BeadChip; SEQ ID NO:68: target sequence of probe cg12323361 from the Infinium HumanMethylation450 BeadChip; SEQ ID NO:69: target sequence of probe cg09644952 from the Infinium HumanMethylation450 BeadChip: SEQ ID NO:70: target sequence of probe cg00697440 from the Infinium HumanMethylation450 BeadChip; SEQ ID NO:71: target sequence of probe cg13069531 from the Infinium HumanMethylation450 BeadChip; SEQ ID NO:72: target sequence of probe cg06327732 from the Infinium HumanMethylation450 BeadChip: SEQ ID NO:73: target sequence of probe cg16331599 from the Infinium HumanMethylation450 BeadChip; SEQ ID NO:74: target sequence of probe cg13617155 from the Infinium HumanMethylation450 BeadChip; SEQ ID NO:75: target sequence of probe cg01436254 from the Infinium HumanMethylation450 BeadChip; SEQ ID NO:76: target sequence of probe cg09410271 from the Infinium HumanMethylation450 BeadChip: SEQ ID NO:77: target sequence of probe cg09838701 from the Infinium HumanMethylation450 BeadChip; to SEQ ID NO:78 CTLA4 qPCR amplicon, unmethylated.

### DETAILED DESCRIPTION OF THE INVENTION

Immune checkpoints are key targets for immunotherapies. A so-called immune checkpoint blockade (ICB) has proven to be particularly effective in the treatment of various malignant diseases. With the help of immune checkpoint inhibitors, the signaling pathways of immunosuppressive immune checkpoints are interrupted so that the body's own immune system can better recognize and fight the malignant cells. However, since immunotherapeutic drugs are usually only effective in certain patients and any effect is often only observed after several months, it is of the utmost importance for clinical practice to have indications in the forefront of a therapy of whether a patient will respond to a particular treatment.

In DE 10 2016 005 947 B3, the inventor of the present invention has already been able to show that in cells of malignant diseases or in T lymphocytes interacting with the cells of the malignant diseases, respectively, the DNA methylation of central immunoregulatory genes such as the PDCD1 gene coding for PD-1 is directly correlated with the expression of the immune checkpoints encoded by these genes. The inventor has also discovered that this correlation allows to predict a patient's response to immunotherapy by analyzing the DNA methylation of these immunoregulatory genes. For example, a response to a pharmaceutical compound that inhibits the PD-1 receptor is more likely if DNA methylation analysis of the corresponding PDCD-1 gene from cells of the malignant disease or from T lymphocytes interacting with cells of the malignant disease indicates that the PD-1 receptor is initially expressed by the cells.

In the course of intensive further research work and extensive clinical studies, the inventor has now been able to show that, surprisingly, DNA methylation of the immunoregulatory genes CTLA4, CD86, CD28, CD80 and ICOS also indicates with particularly high reliability whether a malignant disease responds to an immunotherapy that inhibits a PD-1 immune checkpoint signaling pathway. In this respect, reference is also made to the following examples. This new finding was not expectable from a skilled person's point of view, because these genes code for immune checkpoints that are not directly related to the PD-1 immune checkpoint signaling pathway. Therefore, the new findings of the inventor differ fundamentally from the disclosure and teaching of DE 10 2016 005 947 B3, which is limited to the discovery that a DNA methylation analysis of an immunoregulatory gene is suitable for predicting the response to an immunotherapy directed against the immune checkpoint that is encoded by the same immunoregulatory gene.

Against this background, a first aspect of the present invention is directed to a method for predicting a response of a malignant disease to an immunotherapy designed to inhibit a PD-1 immune checkpoint signaling pathway. In this method, at least a part of an immunoregulatory gene of cells of said malignant disease and/or of immune cells interacting with cells of said malignant disease is subjected to DNA methylation analysis in order to analyze said immunoregulatory gene for DNA methylation. The immunoregulatory gene is selected from the group consisting of CTLA4, CD86, CD28, CD80, ICOS or any combination thereof. On the basis of this DNA methylation analysis, the response of said malignant disease to said immunotherapy is then predicted based on presence, absence and/or level of DNA methylation of said immunoregulatory gene (prediction).

The present invention is particularly characterized by the fact that DNA methylation analysis of the immunoregulatory genes according to the invention is universally suitable for predicting the response behavior of a wide variety of malignant diseases or tumor entities. This discovery by the inventor is in line with the latest state of knowledge of the U.S. Food and Drug Administration, which recently approved for the first time an immunotherapy of cancer diseases based on a common genetic characteristic of the diseases instead of defining the approval on the basis of the diseased organ as was previously standard practice (Chang et al., Appl Immunohistochem Mol Morphol. 2017, Epub ahead of print). The malignant disease may in particular include melanoma, carcinoma, sarcoma, glioblastoma lymphoma and/or leukemia. The carcinoma may be an adenocarcinoma, a squamous cell carcinoma, a small cell carcinoma, a neuroendocrine carcinoma, a renal cell carcinoma, a urothelial carcinoma, a hepatocellular carcinoma, an anal carcinoma, a bronchial carcinoma, an endometrial carcinoma, a cholangiocellular carcinoma, a hepatocellular carcinoma, a testicular carcinoma, a colorectal carcinoma, a carcinoma of the head and neck region, a carcinoma of the esophagus, a stomach carcinoma, a breast carcinoma, a kidney carcinoma, an ovarian carcinoma, a pancreatic carcinoma, a prostate carcinoma, a thyroid carcinoma and/or a cervical carcinoma, for example. A sarcoma may for example be an angiosarcoma, a chondrosarcoma, a Ewing sarcoma, a fibrosarcoma, a Kaposi sarcoma, a liposarcoma, a leiomyosarcoma, a malignant fibrous histiocytoma, a neurogenic sarcoma, an osteosarcoma or a rhabdomyosarcoma. A leukemia can be, for example, acute myeloid leukemia (AML), acute lymphocytic leukemia (ALL), chronic lymphocytic leukemia (CLL), or chronic myeloid leukemia (CML). Lymphoma can be Hodgkin's lymphoma or non-Hodgkin's lymphoma. Non-Hodgkin's lymphoma can be B-cell lymphoma or T-cell lymphoma. In particular, the malignant disease is a malignant melanoma, which may be metastasized.

The immunotherapy preferably comprises a pharmaceutical compound which binds to PD-1, PD-L1 and/or PD-L2 and, through this binding, inhibits the PD-1 immune checkpoint signaling pathway, for example by inhibiting the native interaction between the PD-1 receptor and its ligands. In particular, the immunotherapy or the pharmaceutical compound may comprise an anti-PD-1 antibody, an anti-PD-L1 antibody and/or anti-PD-L2 antibody. It is preferably a monoclonal antibody. The pharmaceutical compound may be selected from nivolumab (BMS-936558, trade name: Opdivo, manufacturer: Bristol-Myers Squibb), pembrolizumab (MK-3475, SCH900475, trade name: Keytruda, manufacturer: Merck/MSD Sharp & Dohme), pidilizumab (CT-011, MDV9300; manufacturer: CureTech Ltd, licensed by Medivation), MGD013 (Macrogenics), AMP-224 (manufacturer: GlaxoSmithKline), MEDI0680 (AMP-514, manufacturer: MedImmune LLC), AUNP-12 (manufacturer: Aurigene Discovery Technologies Ltd.), BMS935559 (MDX-1105, manufacturer: Bristol-Myers Squibb), CA-170 and CA-237 (manufacturer: Curis Inc.), MPDL3280A (manufacturer: Roche), MEDI4736 (manufacturer: AstraZeneca), avelumab (MSB0010718C. manufacturer: Pfizer) and/or rHIgM12B7 (B7-DC cross-linking antibody rHIgM12B7, Mayo Clinic), TSR-042 (manufacturer: Tesaro), SHR-1210 (manufacturer: Jiangsu Hengrui Medicine Co., Ltd.), Sym021 (manufacturer: Symphogen A/S), REGN2810 (manufacturer: Regeneron), JNJ-63723283 (manufacturer: JoJanssen Research & Development, LLC), BGB-A317 (manufacturer: BeiGene). PDR001 (manufacturer: Novartis), JTX-4014 (manufacturer: Jounce Therapeutics), atezolizumab (MPDL3280A, manufacturer: Genentech/Roche), durvalumab (MED14736, MEDI-4736, manufacturer: Medimmune/AstraZeneca), LY3300054 (manufacturer: Lilly), KN035 (manufacturer: Suzhou Alphamab Co. Ltd.), CX-072 (Manufacturer: CytomX Therapeutics) and any combinations thereof.

The DNA methylation analysis can basically be performed with any of the common methods known to the skilled person from the pertinent literature. A suitable method includes, for example, the following steps: A) Providing DNA of the cells of said malignant disease or of the immune cells interacting with cells of said malignant disease; B) converting at least part of the cytosines contained in said DNA from A) into uracil or another base having a base pairing behavior and/or molecular weight distinguishable from cytosine: C) analyzing the DNA obtained from step B) for DNA methylation of said immunoregulatory gene.

The DNA from step A) which is to be analyzed may originate from different sources and may include, for example, cells of the malignant disease or infiltrating immune cells, in particular tumor-infiltrating T lymphocytes, from surgically obtained or biopsied tissue. The (immune) cells may also be obtained from swabs and from aspirates such as rinsing fluids, fine needle aspirates or sputum. The DNA can also be obtained from blood, blood serum and blood plasma, for example in the form of circulating cell-free DNA, exosomal DNA, or in the form of circulating cells of the malignant disease and/or peripheral immune cells from which the DNA is derived. The DNA may also be obtained from other bodily fluids such as lymphatic fluid, urine, pleural effusions or ascites, for example in the form of circulating cell-free DNA or in the form of circulating cells of the malignant disease or circulating immune cells from which the DNA is derived. The DNA can also be obtained from non-preserved (fresh) cells, tissues and body fluids, as well as from fixed cells, tissues and body fluids. The fixation of the (immune) cells, tissues and body fluids can be achieved by precipitating fixatives such as ethanol and other alcohols or by cross-linking fixatives such as formaldehyde. For example, formalin-fixed and paraffin-embedded tissue (FFPET) may be used. The DNA can also be obtained from any combination of these sources. It may also be DNA extracted from the above sources. It is also possible to enrich the DNA, for example by precipitation or extraction. This can be done, for example, with circulating cell-free DNA from the body fluids mentioned above. It is also possible to enrich the (immune) cells, for example by size filtration or via magnetic particles carrying antibodies on their surface, whose antigens are located on the surface of the (immune) cells to be enriched.

This can be useful, for example, for circulating cells of the malignant disease or circulating immune cells from the aforementioned body fluids. Other suitable sources for the DNA to be analyzed are homogenizates of fresh tissue and lysates of fixed tissue. In contrast to conventional prediction methods based on immunohistochemical methods or mRNA expression analysis, a particular advantage of the present invention is that DNA methylation provides particularly robust and accurate prediction results even in the case of conserved sample materials, minute amounts of cells or completely cell-free DNA samples.

In a preferred variant, the DNA therefore comprises circulating cell-free DNA, DNA from exosomes and/or DNA from circulating (immune) cells from a body fluid, so-called “liquid biopsies”. Liquid biopsies currently represent a central area of oncological research. Instead of analyzing the suspicious tissue itself, for example tumor tissue, liquid biopsies analyze a sample of body fluid, for example a blood sample or lymphatic fluid sample. This sample can be used to analyze different substances that originate from the tumor, because circulating cell-free genomic DNA, exosomal DNA, as well as circulating cells or circulating immune cells are released from the tumor into the bloodstream. They may also be immune cells that come from the thymus or a lymph node and are able to interact specifically with the cells of the malignant disease, but do not originate from the tumor itself. It is advantageous to use the method of the present invention for the analysis of liquid biopsies if the tumor or a metastasis cannot be biopsied or if a biopsy would be too dangerous for the patient in the late tumor stage. The present invention is characterized by the fact that the DNA methylation of immunoregulatory genes can be measured very reliably in bodily fluids, where conventional determination of expression of immunoregulatory genes using mRNA or immunohistochemistry is difficult or even impossible.

The conversion of the DNA in step B) can in principle be carried out using any of the methods known in the state of the art and suitable for this purpose. It is typically a chemical or enzymatic conversion, for example by contacting the DNA with bisulfite, for example sodium bisulfite or ammonium bisulfite.

If necessary, the DNA may be purified after the conversion in step B) and prior to analyzing the DNA methylation in step C). Suitable purification methods and protocols are known to the skilled person and may include DNA extraction, precipitation or polymer-mediated enrichment, for example. In this respect, reference is also made to the embodiments described above.

The DNA methylation analysis is used to determine the presence, absence or level of DNA methylation in the analyzed part of the immunoregulatory gene. The analyzed part thus contains at least one CpG dinucleotide which is analyzed for DNA methylation, preferably several CpG dinucleotides which are analyzed for DNA methylation. Presence of DNA methylation therefore means that at least one methylated CpG dinucleotide is detected in the analyzed part of the immunoregulatory gene. Absence of DNA methylation means that no methylation is detectable in any of the CpG dinucleotides contained in said part. Determining the level of DNA methylation of the immunoregulatory gene may comprise analyzing several CpG dinucleotides for DNA methylation which are contained in the analyzed part. The determination of the level of DNA methylation of the immunoregulatory gene may also comprise an analysis of the same CpG dinucleotide for DNA methylation in a plurality of gene copies of the immunoregulatory gene. Combinations of these variants are also possible.

The analysis of the DNA methylation of the immunoregulatory gene in step C) is not subject to any particular restrictions. Suitable methods can easily be determined by the skilled person on the basis of this disclosure. In this respect, reference is also made to the laboratory manuals mentioned hereinabove. In a preferred variant, a polymerase chain reaction (PCR) is initially carried out with oligonucleotides, so-called primers, which is designed to amplify a section of the DNA converted in step B), which comprises the part of the immunoregulatory gene or the at least one CpG dinucleotide to be analyzed. Subsequently, at least a part of the amplification product is sequenced, for example by a Sanger sequencing, pyrosequencing, mass spectrometric sequencing or a sequencing of the second or third generation, which are also referred to as “massive parallel sequencing”, “next generation sequencing” (NGS) or nanopore sequencing. It is also possible to carry out hybridization with methylation-specific oligonucleotides (probes) following the PCR, for example in the form of a DNA microarray. DNA methylation can also be determined by quantitative real-time PCR (qPCR), optionally followed by a melting curve analysis. In particular, quantitative real-time PCR can be performed with methylation-specific primers as described in WO 1997/046705 A1 and/or methylation-specific blocker oligonucleotides as described in WO 2002/072880 A2. In a preferred variant, methylation-specific detection probes are used.

In yet other preferred variants, a PCR can be omitted, for example in the case of “Whole Genome Shotgun Bisulfite Sequencing” (WGSBS) or direct nanopore sequencing. In WGSBS, the DNA is fragmented, followed by ligation of adapters to the DNA fragments. The adapters can subsequently be used for amplification and sequencing. It can also be possible to skip the fragmentation step in the WGSBS, since the DNA may already be fragmented, for example, due to conversion by bisulfite treatment. Protocols for performing a WGSBS are readily available for the skilled person (Johnson, M. D. et al., Curr. Protoc. Mol. Biol., 2012. 99, 21.23.1-21.23.28; Lister. R. et al., Nature, 2009, 462, 315-322, Berman, B. P. et al., Nat. Genet., 2011, 44, 40-46).

In another preferred variant, hybridization with specific oligonucleotides (probes) can take place prior to PCR amplification. In the case of binding, the oligonucleotides are ligated and subsequently amplified by PCR. Suitable methods and protocols, such as “multiplex ligation dependent probe amplification” (MLPA) are readily available for the skilled person without difficulties, for example in “PCR Mutation Detection Protocols” by B. D. M. Theophilus and R. Rapley, 2nd Edition, 2011, Springer.

In another preferred variant, the DNA methylation analysis is performed using the Infinium HumanMethylation450 BeadChip. Suitable protocols can be found, for example, in the chapter “Determination of DNA Methylation Levels Using Illumina HumanMethylation450 BeadChips” by M. A. Carless, which can be found in the book “Chromatin Protocols” by S. P. Chellappan, Volume 1288, 2015, of the book series “Methods in Molecular Biology”, Springer Science+Business Media New York. Further suitable protocols are described in the following examples.

The inventor found out that the response of the malignant disease to the immunotherapy is the more likely the lower the level of DNA methylation of the immunoregulatory gene.

Consequently, the response of the malignant disease to immunotherapy is particularly likely when the DNA methylation of the immunoregulatory gene is close to zero, in particular when DNA methylation is actually absent or at least virtually absent due to technical detection limits.

It is therefore advantageous if the DNA methylation analysis is carried out under conditions which allow a quantitative determination of the DNA methylation of the immunoregulatory gene. If, for example, the DNA methylation analysis comprises gene copies of the immunoregulatory gene of several cells of the malignant disease and/or of several of the immune cells interacting with the cells of the malignant disease, a proportion of said gene copies of the immunoregulatory gene which contain the DNA methylation can be determined. For this purpose, the number or amount of methylated gene copies of the immunoregulatory gene may be correlated with the total number or amount of gene copies of the immunoregulatory gene being analyzed. In this way, the response of the malignant disease to the immunotherapy can be predicted with even greater accuracy and can be likely, for example, if less than or equal to 40%, less than or equal to 35%, less than or equal to 30%, less than or equal to 25%, less than or equal to 20%, less than or equal to 15%, less than or equal to 10% or less than or equal to 5% of the gene copies of the immunoregulatory gene contain the DNA methylation. In yet other variants, the malignant disease may be likely not to respond to the immunotherapy if more than 30%, more than 35%, more than 40% or more than 45% of the gene copies of the immunoregulatory gene contain the DNA methylation, or generally in the presence of DNA methylation.

The immune cells interacting with cells of the malignant disease can be T lymphocytes, B lymphocytes, antigen-presenting cells or natural killer cells (NKs). Any combination of these immune cells is also possible. In certain variants, the immune cells include tumor-infiltrating T lymphocytes and/or B lymphocytes, in particular tumor-infiltrating CD8+lymphocytes and/or regulatory T lymphocytes (Tregs). However, it can also be peripheral and/or lymphatic T lymphocytes, B-lymphocytes, antigen-presenting cells and/or NKs.

The DNA methylation analysis can comprise one or more parts or one or more CpG dinucleotides within and surrounding the immunoregulatory gene. Preferably, the DNA methylation analysis comprises at least a part of a regulatory gene region, in particular of a transcription factor binding site, of a promoter, of a CpG island, of a silencer, of an enhancer, or of a CTCF binding site. The DNA methylation analysis can also comprise at least part of a sequence coding for a transcript of the immunoregulatory gene. Any combination of the aforementioned parts is also possible. Enhancers may be present as distal enhancers remote from the gene, for example. Enhancers can also be located near the gene and are then known as proximal enhancers. Regulatory gene regions are well known to the skilled person and are described, for example, in “Gene Control” by D. S. Latchman, 2nd Edition, 2015, Garyland Science, Taylor & Francis Group. LLC. For example, those parts or CpG dinucleotides whose state of methylation correlates with the transcriptional activity or the expression of the immunoregulatory gene are also suitable for carrying out the method according to the invention.

The transcriptional activity can be identified, for example, by an altered chromatin structure. So-called “open chromatin” can be associated with a high transcriptional activity of a gene, as described for example in “Genetics” by W. Janning and E. Kunst, 2004, Georg Thieme Verlag, Stuttgart and New York. Areas of “open chromatin” are therefore suitable for the DNA methylation analysis of the present invention.

The determination of regulatory genetic elements is easily possible for the skilled person using suitable databases. For example, in the database “Ensembl” such regulatory elements are annotated, as described in “The Ensembl Regulatory Build” by D. R Zerbino, S. P. Wilder, N. Johnson, T. Juettemann and P. R. Flicek, 2015, Genome Biology, Issue 16, doi:10.1186/s13059-015-0621-5.

A suitable primary sequence of the human genome, on the basis of which suitable and preferred regions and sequences of immunoregulatory genes for the DNA methylation analysis of the present invention can be determined, is for instance the human genome version of the Genome Reference Consortium Human Build 38 (GRCh38) or Genome Reference Consortium Human Build 38 patch release 10 (GRCh38) as of 15 Oct. 2017. In the following, regions of the genome are referred to in the notation “chromosome number:position of the first base of the region-position of the last base of the region”, e. g. “2:203583059-203583108” for the region from base 203583059 to base 203583108 of chromosome 2.

The DNA methylation analysis of the CTL44 gene preferably comprises at least a part of one or more of the following regions: a region comprising the CTLA4 gene and the adjacent co-methylated genes CD28 and ICOS (2:203551590-204126647, SEQ ID NO: 1), a region encoding the transcripts (2:203867786-203873960), a promoter (2:203866174-203868926 and 2:203869477-203874095), an enhancer (2:203874152-203875266, 2:203876672-203878051 and 2:203879313-203881585), a region between CTLA4 and the adjacent immunoregulatory gene ICOS (2:203872383-203939876), a region between CTLA4 and the adjacent immunoregulatory gene CD28 (2:203738066-203867984), one or more regions selected from SEQ ID NO:51, SEQ ID NO:27, SEQ ID NO:48, SEQ ID NO:28, SEQ ID NO:29, SEQ ID NO:50. SEQ ID NO:32, SEQ ID NO:33, SEQ ID NO:30, SEQ ID NO:49, SEQ ID NO:31 and SEQ ID NO:52.

The DNA methylation analysis of the CD28 gene preferably comprises at least a part of one or more of the following regions: a region comprising a coding sequence of CD28 and a promoter (2:203551590-203754454), a region comprising the coding sequence (2:203706475-203738912) and a region comprising a promoter (2:203677265-203707326).

The DNA methylation analysis of the ICOS gene preferably includes at least a part of one or more of the following regions: a region coding for a transcript (2:203936748-203961577), a promoter (2:203934590-203941036 and 2:203948548-203953636), an enhancer (2:203931099-203937863 and 2:203940518-203949061), one or more regions selected from SEQ ID NO:52, SEQ ID NO:53, SEQ ID NO:34, SEQ ID NO:54, SEQ ID NO:36. SEQ ID NO:56, SEQ ID NO:35 and SEQ ID NO:55.

The DNA methylation analysis of the CD86 gene preferably comprises at least part of one or more of the following regions: a region comprising a coding sequence of CD86 and a promoter (3:122039741-122154807, SEQ ID NO:3), a region coding for transcripts (3:122054701-122121475), a promoter (3:122054261-122061082, 3:122073513-122079893 and 3:122087703-122093314), an enhancer (3:122098924-122104974), one or more regions selected from SEQ ID NO:65, SEQ ID NO:67, SEQ ID NO:66, SEQ ID NO:70, SEQ ID NO:72, SEQ ID NO:69, SEQ ID NO:75, SEQ ID NO:73, SEQ ID NO:74, SEQ ID NO:71, SEQ ID NO:68, SEQ ID NO:76, SEQ ID NO:77.

The DNA methylation analysis of the CD80 gene preferably comprises at least part of one or more of the following regions: a region comprising a coding sequence of CD80 and a promoter (3:119523584-119573836, SEQ ID NO:2), a region coding for transcripts (3:119524293-119559602), a promoter (3:119554042-119563668 and 3:119568227-119573274), an enhancer (3:119563379-119568778, 3:119538188-119543511 and 3:119545840-119554325), one or more regions selected from SEQ ID NO:57, SEQ ID NO:61, SEQ ID NO:62, SEQ ID NO:60, SEQ ID NO:59, SEQ ID NO:64, SEQ ID NO:58 and SEQ ID NO:63.

In a preferred variant, the DNA methylation analysis comprises at least part of CTLA4. In another preferred variant, the DNA methylation analysis comprises at least part of CD86. In yet another preferred variant, the DNA methylation analysis comprises at least part of CD28. In yet another preferred variant, the DNA methylation analysis comprises at least part of CD80. In yet another preferred variant, the DNA methylation analysis comprises at least part of ICOS. In particularly preferred variants, the DNA methylation analysis comprises at least a part of each of at least two of the immunoregulatory genes. In this way, a reliable prediction of the response of the malignant disease is achieved, in particular if only small amounts of DNA are available for the DNA methylation analysis. In this respect, reference is also made to the following example 2. Preferably, the DNA methylation analysis comprises at least part of CTLA4 and at least part of at least one other of the immunoregulatory genes.

The DNA methylation analysis can also include other immunoregulatory genes in addition to the above-mentioned genes in order to achieve even more precise subtyping of patients by combining the results. In particular, the DNA methylation analysis can additionally comprise at least a part of PDCD1, CD274 and/or PDCD1LG2, which code for the immune checkpoints PD-1, PD-L1 and PD-L2. For example, the DNA methylation analysis may comprise at least part of CTLA4 and at least part of PDCD1. CD274 and/or PDCD1LG2. The DNA methylation analysis may also comprise at least part of CTLA4 and at least part of PDCD1, CD274, PDCD1LG2, CD86. CD28. CD80 and/or ICOS.

The DNA methylation analysis of the PDCD1 gene preferably includes at least part of one or more of the following regions: a region coding for a transcript (2:241849881-241858908), a region with open chromatin (2:241849051-241853001 and 2:241861820-241862593), an enhancer (2:241852997-241855201), a CTCF binding site (2:241859081-241860074), a promoter (2:241856912-241861429 and 2:241862929-241865230).

The DNA methylation analysis of the CD274 gene preferably includes at least a part of one or more of the following regions: a region coding for a transcript (9:5450503-5470566), a promoter (9:5445402-5456799 and 9:5458041-5461360), an enhancer (9:5457122-5457702, 9:5463574-5468340, 9:5440647-5441785 and 9:5472191-5473149), a CTCF binding site (9:5440970-5441435 and 9:5446325-5446870).

The DNA methylation analysis of the PDCD1LG2 gene preferably includes at least part of one or more of the following regions:

a region coding for a transcript (9:5510570-5571254), a promoter (9:5507688-5523442, 9:5491444-5503289, 9:5528150-5534251 and 9:5547972-5571492), an enhancer (9:5479110-5491616, 9:5522642-5528253, 9:5534822-5547690 and 9:5572730-5580962), a region upstream of the coding sequence (9:5496357-5510570).

The aforementioned features and preferred embodiments of the first aspect are also fully incorporated in the following aspects of the invention but are not specifically discussed again hereinafter in order to avoid repetition.

In personalized oncological medicine, therapies are adapted to patients with specific types of cancer. A high response rate of patients to a targeted immunotherapy with a PD-1 immune checkpoint inhibitor can only be achieved if patients with corresponding sensitive diseases are reliably identified and specifically selected for the treatment. In this sense, a second aspect of the present invention provides a method for selecting a patient suffering from a malignant disease for an immunotherapy designed to inhibit a PD-1 immune checkpoint signaling pathway. The method comprises the following steps: A) providing cells of said malignant disease and/or immune cells interacting with cells of said malignant disease from said patient. B) performing a DNA methylation analysis of at least a part of an immunoregulatory gene selected from CTLA4, CD86, CD28, CD80 and/or ICOS from said cells of the malignant disease and/or said immune cells interacting with cells of the malignant disease from step A), C) selecting the patient for said immunotherapy on the basis of the presence, absence and/or level of DNA methylation of said immunoregulatory gene determined in B).

In particular, the patient is selected if the DNA methylation of said immunoregulatory gene indicates that the malignant disease is likely to respond to said immunotherapy. In this respect, reference is made to the corresponding embodiments of the first aspect.

According to a third aspect of the present invention, a DNA methylation analysis of at least a part of an immunoregulatory gene selected from CTLA4, CD86, CD28, CD80 and/or ICOS of cells of a malignant disease and/or of immune cells interacting with cells of the malignant disease can be used in different ways in personalized medicine. Possible uses are, for example, use for predicting a response of said malignant disease to an immunotherapy, use for individualized selection of an immunotherapy for said malignant disease, and/or use for selecting a patient suffering from said malignant disease for an immunotherapy, wherein said immunotherapy is designed to inhibit a PD-1 immune checkpoint signaling pathway.

A fourth aspect of the invention provides use of the presence, absence or level of DNA methylation of at least a part of an immunoregulatory gene selected from CTLA4, CD86, CD28, CD80 and/or ICOS of cells of a malignant disease and/or of immune cells interacting with cells of the malignant disease as a biomarker for predicting a response of said malignant disease to an immunotherapy, for the individualized selection of an immunotherapy for said malignant disease, and/or for selecting a patient suffering from said malignant disease for an immunotherapy, wherein said immunotherapy is in each case designed to inhibit a PD-1 immune checkpoint signaling pathway.

In the aforementioned uses, the immunotherapy may also be additionally designed to inhibit a CTLA4 immune checkpoint signaling pathway. In his clinical studies, the inventor not only recognized that the DNA methylation analysis of the genes of the present invention is equally reliable in indicating a probable response of the malignant disease to such CTLA4 immune checkpoint signaling pathway inhibiting immunotherapies, but also that the combination of PD-1 and CTLA4 immune checkpoint inhibition in one immunotherapy can be associated with particular treatment success. The present invention is therefore distinguished by the fact that it can provide the clinician with at least three treatment options using a single diagnostic test determining the presence, absence or level of DNA methylation of the immunoregulatory gene: a selective inhibition of the PD-1 immune checkpoint pathway (for example as monotherapy), a combined inhibition of the PD-1 and CTLA4 immune checkpoint signaling pathways as well as a sequential inhibition of first the PD-1 immune checkpoint signaling pathway and then the CTLA4 immune checkpoint signaling pathway. The latter option can be useful, for example, if the inhibition of the PD-1 immunocheckpoint signaling pathway causes severe side effects in a patient and must therefore be discontinued, or if the PD-1 inhibition is no longer effective and the patient shows disease progression.

The immunotherapy designed to inhibit the CTLA4 immune checkpoint signaling pathway preferably comprises a pharmaceutical compound that inhibits the CTLA4 immune checkpoint signaling pathway by binding of the pharmaceutical compound to CTLA4, CD80, CD86 or CD28. The immunotherapy or the pharmaceutical compound may for example comprise an anti-CTLA4 antibody, an anti-CD80 antibody, an anti-CD86 antibody and/or an anti-CD28 antibody. In particular, these antibodies can be monoclonal antibodies.

A fifth aspect of the invention provides a kit for carrying out the method according to the first or second aspect or for the use according to the third or fourth aspect of the present invention, respectively. The kit comprises reagents for DNA methylation analysis of at least part of an immunoregulatory gene selected from CTLA4, CD86, CD28, CD80 and/or ICOS of cells of a malignant disease and/or of immune cells interacting with cells of the malignant disease, in order to determine presence, absence and/or level of DNA methylation of said immunoregulatory gene. The kit may also include instructions for predicting the response of the malignant disease to said immunotherapy based on the presence, absence and/or level of DNA methylation of said immunoregulatory gene.

In particular, the kit contains at least one first oligonucleotide pair for the DNA methylation analysis, which is adapted to hybridize to a sequence of the immunoregulatory gene in DNA from said cells of the malignant disease and/or from said immune cells after cytosines contained in said DNA have been converted into uracil or another base having a base pairing behavior and/or molecular weight distinguishable from cytosine, in order to amplify and/or detect said sequence. At least one of the oligonucleotides may be designed to distinguish between converted methylated and converted unmethylated DNA, so that the sequence is amplified in a methylation-dependent manner. For this purpose, the oligonucleotide may be reverse complementary to a binding sequence containing at least one CpG dinucleotide to be analyzed.

For example, the oligonucleotide can be reverse complementary to the binding sequence if the cytosine in the CpG dinucleotide has been converted, i. e. was originally unmethylated. Alternatively, the oligonucleotide can be reverse complementary to the binding sequence if the cytosine in the CpG was not converted, i. e. was originally methylated. In this way is achieved that an amplification only occurs if the sequence is methylated or unmethylated, respectively.

It is also possible that the oligonucleotides are designed to amplify the sequence independently of DNA methylation. Preferably, the oligonucleotides are in this case reverse complementary to binding sequences that do not contain CpG dinucleotides to be analyzed. Preferably, the CpG dinucleotides to be analyzed are located between the binding sequences of the oligonucleotides. The kit may additionally contain hybridization probes which differentiate between a converted methylated sequence and a converted unmethylated sequence so that the amplified sequence is detected in a methylation-dependent manner. The level of DNA methylation can then be determined from the signal ratio of the probes.

Furthermore, the kit can comprise at least a second pair of oligonucleotides adapted to hybridize to a sequence of the converted DNA which does not contain CpG dinucleotides, in order to amplify and/or detect the sequence methylation-independently. With the aid of an oligonucleotide pair designed in this way, it is for instance possible to determine the total number or total amount of genome copies or gene copies, in particular of gene copies of the immunoregulatory gene, present in the converted DNA. In this way, the relative proportion of those gene copies of the immunoregulatory gene which contain the DNA methylation can be determined, as described in the corresponding embodiments of the first aspect. In this respect, reference is also made to the first example.

In preferred variants, the kit contains two or more first oligonucleotide pairs adapted to hybridize to sequences of at least two different immunoregulatory genes selected from CTLA4, CD86, CD28, CD80 and/or ICOS in the converted DNA to amplify and/or detect the sequences for the DNA methylation analysis. By the combined DNA methylation analysis of at least two of the immunoregulatory genes of the present invention, a particularly robust measurement and thus a particularly reliable prediction of the response to the immunotherapy is achieved. In this respect, reference is also made to the second example.

Preferred regions and sequences of the immunoregulatory gene to be amplified and/or detected by the oligonucleotide pairs correspond to those of the first aspect.

The kit preferably includes instructions for use for carrying out the method according to the first and/or second aspect and/or for the use according to the third or fourth aspect.

All aforementioned aspects of the present invention concern deductive steps in connection with a preceding in viro method, so that no technical step essential to the invention takes place on the human or animal body.

In principle, however, it is also possible to implement the method of the present invention into a tailored treatment approach for a patient with a malignant disease in order to improve the probability of a therapeutic response. Consequently, a sixth aspect of the invention provides a method for an immunotherapeutic treatment of a patient suffering from a malignant disease with a pharmaceutical compound designed to inhibit a PD-1 immune checkpoint signaling pathway. The method comprises in step I), prior to and/or during immunotherapeutic treatment, performing a DNA methylation analysis of at least a part of an immunoregulatory gene selected from CTLA4, CD86, CD28, CD80 and/or ICOS from cells of said malignant disease and/or immune cells interacting with cells of said malignant disease and predicting the response of said malignant disease to said pharmaceutical compound based on presence, absence and/or level of DNA methylation of said immunoregulatory gene. Subsequently, in step II) the pharmaceutical compound is administered if step I) indicates that the malignant disease is likely to respond to immunotherapeutic treatment with said pharmaceutical compound. However, if step I) indicates that the malignant disease is unlikely to respond or is no longer likely to respond to the immunotherapeutic treatment with said pharmaceutical compound, as an alternative to step II) the pharmaceutical compound is not administered, or administration of the pharmaceutical compound is discontinued or reduced (step III). Regarding the probability of response, reference is made to the embodiments of the first aspect. In this way, the present invention contributes with diagnostic methods to a better typing of malignant disease and thus enables a tailored treatment with an improved benefit-risk ratio.

During treatment, it is possible to administer in step II) in addition to the pharmaceutical compound and/or in step III) instead of the pharmaceutical compound another or different pharmaceutical compound, which is designed to inhibit a CTLA4 immune checkpoint signaling pathway. As already described in the fourth aspect, a particular advantage of the present invention is that the clinician can be provided with both complementary and alternative immunotherapeutic treatment options with the aid of a single diagnostic test: a selective inhibition of the PD-1 immunocheckpoint pathway or alternatively the CTLA4 immunocheckpoint pathway, a combined inhibition of the PD-1 and CTLA4 immunocheckpoint pathways, and a sequential inhibition of first the PD-1 immunocheckpoint pathway and then the CTLA4 immunocheckpoint pathway. In this way, the clinician can make more targeted and efficient use of the treatment options for the malignant disease.

The additional or different pharmaceutical compound can comprise, for example, an anti-CTLA4 antibody, an anti-CD80 antibody, an anti-CD86 antibody and/or an anti-CD28 antibody. In particular, it can be a monoclonal antibody.

### EXAMPLES

In the following, the invention is described in more detail by way of examples and experimental results. These examples are intended for explanation only and not as a limitation to specific details.

### Example 1: Clinical Study for Predicting the Response of a Malignant Disease to Inhibition of the PD-1 Immune Checkpoint Pathway Based on DNA Methylation of CTLA4

The studied patient cohort included a total of 50 patients diagnosed with metastatic malignant melanomas. Before the start of the immunotherapeutic treatment, tumor tissue samples were taken from the patients, fixed with formalin and embedded in paraffin. Patients were treated with anti-PD-1 immunocheckpoint blockade using pembrolizumab or nivolumab between October 2014 and April 2017.

For the DNA methylation analysis, thin sections from the tumor tissue samples with a thickness of 10 μm were first prepared and then mounted on glass slides. Using a HE section, the tumor areas were identified by pathological examination and scraped off the glass slides with a scalpel for further treatment. Bisulfite-converted DNA was prepared from the tumor areas using the innuCONVERT Bisulfite All-In-One Kit (Analytik Jena, Jena, Germany) according to the manufacturer's instructions. The total amount of converted DNA was then quantified using a NanoDrop ND-1000 spectrophotometer (Thermo Fisher Scientific, Waltham, Mass., USA).

In the next step, a DNA methylation analysis was performed, for instance by amplifying and simultaneously quantifying a portion of the CTLA4 gene locus methylation-specifically using a quantitative real-time PCR. For example, a duplex PCR was used in which, in addition to DNA methylation of CTLA4, the total DNA, i. e. the total amount and/or total number of genome copies of the converted DNA, was determined within the same reaction. The methylation-specific amplification of the CTLA4 locus was achieved using primers of the sequences SEQ ID NO:9 and SEQ ID NO: 10, which amplify the sequence resulting from bisulfite conversion of the sequence SEQ ID NO: 13. In the case of complete methylation, this converted region in the genome has the sequence SEQ ID NO: 12. The methylation-specific detection was achieved with a probe of the sequence SEQ ID NO: 11, which carried the fluorescent dye 6-FAM at 5′ and the quencher BHQ-1 at 3′. For the determination of total DNA, a locus in the ACTB gene was amplified in a methylation independent manner. This locus has the sequence with SEQ ID NO:8 in the genome and has the sequence with SEQ ID NO:7 after conversion by bisulfite. This sequence was amplified using primers with the sequences SEQ ID NO:4 and SEQ ID NO:5. Sequence-specific detection of the amplification product was achieved with the probe of sequence SEQ ID NO:6, which carried the fluorescent dye Atto 647N at 5′ and the quencher BHQ-2 at 3′.

The real-time PCR was performed in 20 μl PCR reactions in three independent measurements each, using for example the following suitable reaction composition: 35 mM Tris-HCl. pH 8.4, 6 mM MgCl₂, 50 mM KCl, 4% glycerol, 0.25 mM of each dNTP (dTTP, dATP, dGTP, dCTP), 2 U FastStart Taq DNA polymerase (Roche Applied Science, Penzberg, Germany), 0.4 μM of each primer and 0.2 μM of each detection probe. The qPCR was performed, for example, using an AB 7500 Fast Real-Time PCR System (Life Technologies Corporation, Carlsbad, Calif., USA). A suitable temperature profile included for instance the following steps: 20 min at 95° C., followed by 45 cycles of 45 s each at 56° C. and 15 s at 95° C.

The amount of CTLA4 methylation in the converted DNA was calculated using the DeltaDelta-CT method and expressed as a percentage in relation to a standard DNA with 100% methylation. The standard DNA used was artificially methylated DNA (CpGenome™ Universal Methylated DNA; Merck Millipore, Darmstadt. Germany), which had previously been converted using the innuCONVERT Bisulfite All-In-One Kit in accordance with the manufacturer's instructions.

The response to immunotherapy was retrospectively evaluated according to the immune-related response evaluation criteria in solid tumors (irRECIST). Death was considered the endpoint for survival analysis. Survival duration was defined as the time from the first administration of the immune checkpoint inhibitor until the time of death. Survival data were used for Kaplan-Meyer analysis with log-rank test. Hazard ratios were calculated using the univariate Cox proportional hazard model and DNA methylation values of CTLA4 were logarithmized to base 2. Comparisons were performed by one-sided ANOVA and Bonferroni post-hoc tests. Categorical variables were tested using the Chi-square test (χ²-test). For statistical analysis, SPSS version 23.0 was used (SPSS Inc., Chicago, Ill., USA).

FIG. 1 shows a boxplot evaluation of the relationship between relative CTLA4 methylation (in %, y-axis) and the response of patients grouped according to the irRECIST criteria (x-axis). Mean CTLA4 methylation was 46.1% (95% confidence interval CI: 31.2-61.0) in the group with progressive disease, 21.6% (95% CI: 11.2-31.9) in the group with stable disease, 7.6% (95% CI: 1.1-14.2) in the group with partial remission and 4.9% in the group with complete remission. These results show clearly and with high statistical significance (p=0.018) that low DNA methylation of the CTLA4 gene is associated with a response of the malignant disease to the immunotherapy aimed at inhibiting the PD-1 immune checkpoint pathway. Furthermore, the results show that the strength of the response also correlates with the level or the degree of DNA methylation of the CTLA4 gene. Accordingly, the lesser the CTLA4 gene is methylated, the better the response.

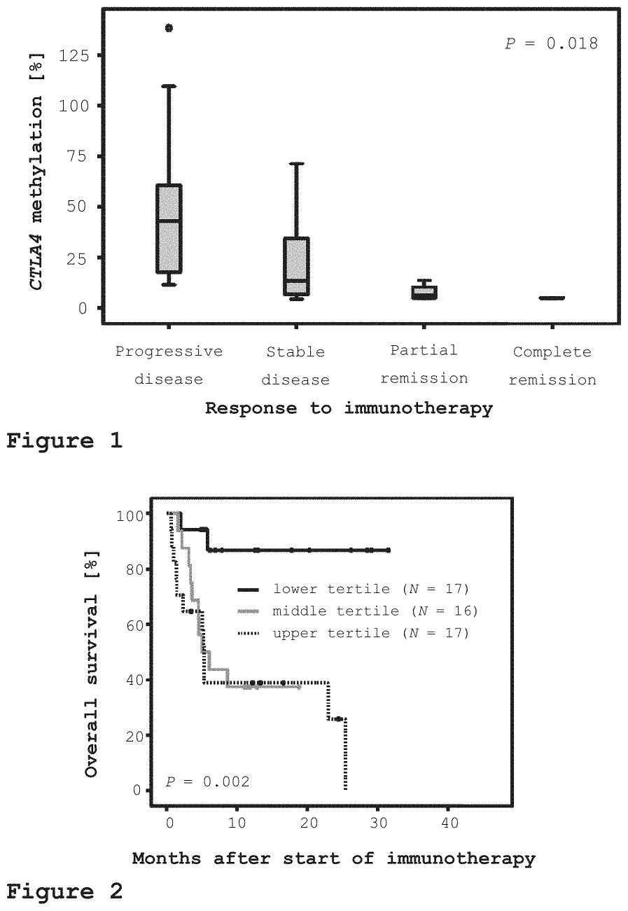

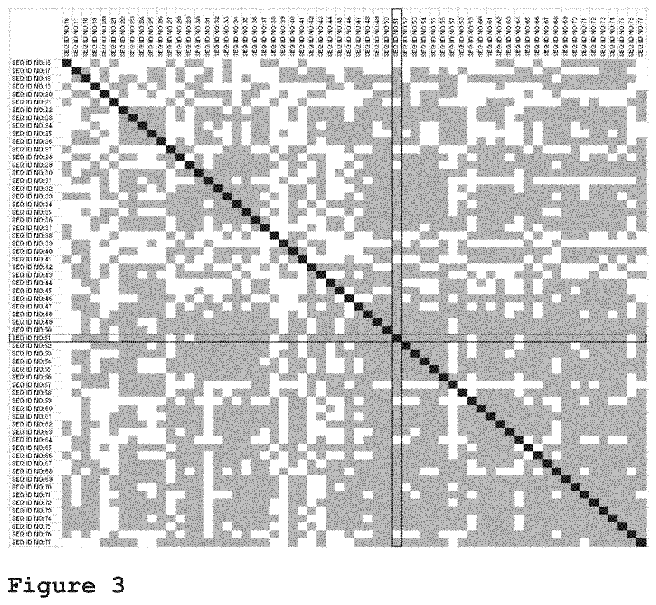

FIG. 2 shows an additional Kaplan-Meier analysis of the overall survival of the 50 patients with malignant melanomas during the immunotherapy. Patients were categorized according to a three-level evaluation using CTL44 methylation tertiles, as is common in pathological classification. The analysis shows a highly significant (p=0.002) prolonged survival of the patients in the lower CTLA4 methylation tertile compared to the middle and upper CTLA4 methylation tertile. Of the 17 patients with the lowest CTLA4 methylation in the tumor, which form the lower CTLA4 methylation tertile, more than 80% were still alive or censored 30 months after the start of the immunotherapy. Of the 17 patients with the highest CTLA4 methylation in the tumor, which form the upper CTL44 methylation tertile, and the 16 patients of the middle CTLA4 methylation tertile, less than 40% survived longer than 12 months after the start of immunotherapy.

Thus, the inventor was able to show for the first time that DNA methylation analysis of the immunoregulatory gene CTL44 of cells of a malignant disease and/or of immune cells interacting with said cells of the malignant disease allows a prediction of the response of the malignant disease to an immunotherapy designed to inhibit a PD-1 immune checkpoint signaling pathway with high reliability. Accordingly, the presence, absence or level of DNA methylation of CTLA4 has been shown to be a reliable biomarker for predicting the response of malignant disease to such immunotherapy.

### Example 2: Determination of Co-Methylation of CTLA4, CD86, CD28, CD80 and ICOS in Various Malignant Diseases

In the framework of the present invention it was further recognized that the response of a patient to immunotherapy can be determined not only by DNA methylation analysis of CTLA4, but also by DNA methylation analysis of an immunoregulatory gene whose DNA methylation is correlated with the DNA methylation of CTLA4. Particularly suitable for this purpose were the immunoregulatory genes CD28, CD80, CD86 and/or ICOS. These genes are functionally related to CTLA4. Furthermore, CD28 and ICOS are also located in the genome in close proximity to CTLA4.

The co-methylation of CTLA4, CD28, CD80, CD86 and ICOS was investigated using a genome-wide DNA methylation analysis, for which, for example, the Infinium HumanMethylation450 BeadChip (Illumina, Inc., San Diego, Calif., USA) used according to the manufacturer's specifications was suitable. The HumanMethylation450 BeadChip raw data was generated as described by the TCGA Research Network (http://cancergenome.nih.govl). In total, raw data from 419 urothelial carcinomas of the bladder, 797 breast cancer tumors, 530 squamous cell carcinomas of the head and neck, 325 clear cell renal cell carcinomas, 475 adenocarcinomas of the lung, 370 squamous cell carcinomas of the lung, 265 sarcomas and 473 cutaneous melanomas were used and analyzed retrospectively.

The DNA methylation analysis included different regions of the genes, which were covered by the beads of the HumanMethylation450 BeadChip: SEQ ID NO: 16 to SEQ ID NO:21 and SEQ ID NO:37 to SEQ ID NO:42 detect DNA methylation immediately upstream of the CD28 gene. SEQ ID NO:22 to SEQ ID NO:26 and SEQ ID NO:43 to SEQ ID NO:47 detect DNA methylation in the coding region of CD28, SEQ ID NO:27 to SEQ ID NO:29 and SEQ ID NO:48 detect DNA methylation in a region between CD28 and CTL44. SEQ ID NO:30 to SEQ ID NO:33, SEQ ID NO:49 and SEQ ID NO:50 detect DNA methylation in the coding region of the CTLA4 gene. SEQ ID NO:52 allows DNA methylation analysis of a region between CTLA4 and ICOS. SEQ ID NO:34 to SEQ ID NO:36 and SEQ ID NO:53 to SEQ ID NO:56 detect DNA methylation of a coding region of the ICOS gene. SEQ ID NO:57 to SEQ ID NO:64 detect DNA methylation in the coding region and the promoter of the CD80 gene. SEQ ID NO:65 to SEQ ID NO:77 detect DNA methylation in the coding region and the promoter of the CD86 gene. The sequence listing shows which beads of the HumanMethylation450 BeadChip were used for the DNA methylation analysis of the respective sequences.

The co-methylation of these gene loci was determined with respect to the DNA methylation of the sequence SEQ ID NO:51 (Bead cg08460026) of the CTLA4 gene. This contains the same four CpG dinucleotides as SEQ ID NO: 13, which were examined in example 1 by means of quantitative real-time PCR. This allows a direct correlation with the results shown in example 1 for predicting the response of the malignant disease to the immunotherapy.

First, a methylation value was calculated from the HumanMethylation450 BeadChip raw data for each of the considered bead pairs and for each patient sample. For this purpose, the signal of the bead of a pair that binds to the methylated variant (S_M) was related to the signal of the bead of the pair that binds to the unmethylated DNA (S_U). A bead comprises a bound oligonucleotide and is also called a probe herein. The calculation of the DNA methylation based on this relation was done according to the equation: Methylation=(intensity probe S_M)/((intensity probe S_M)+(intensity probe S_U)).

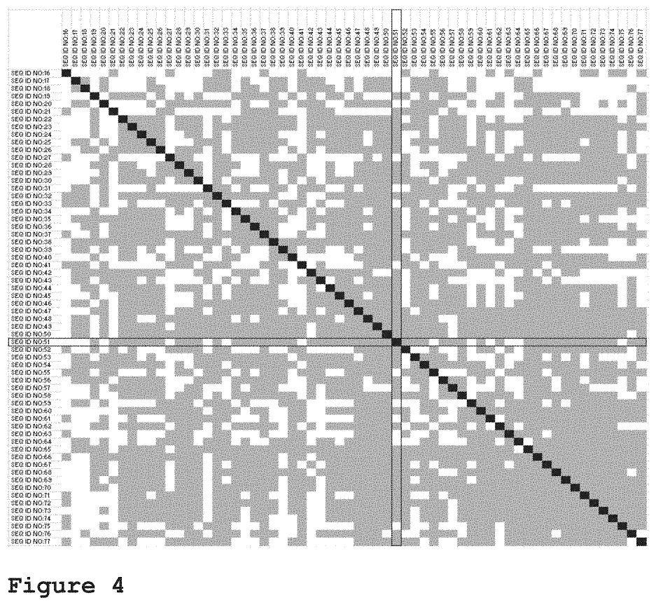

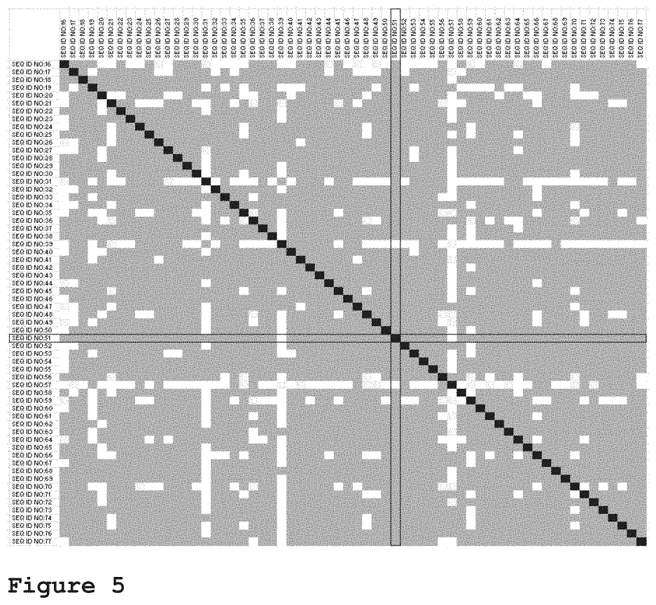

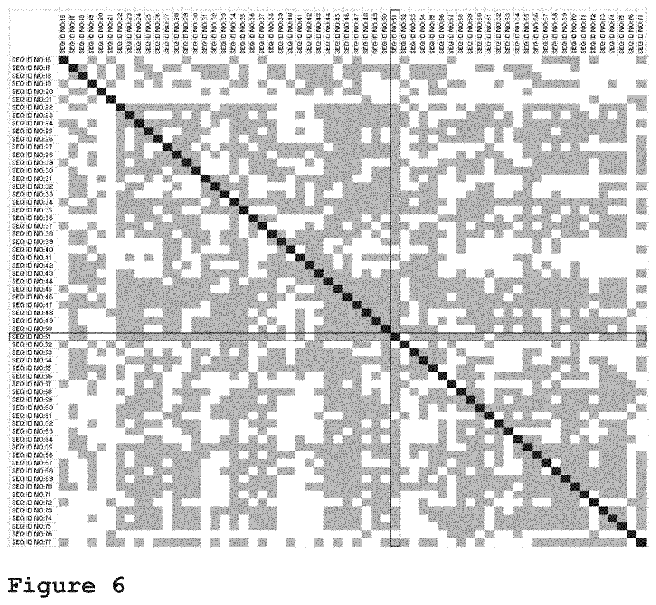

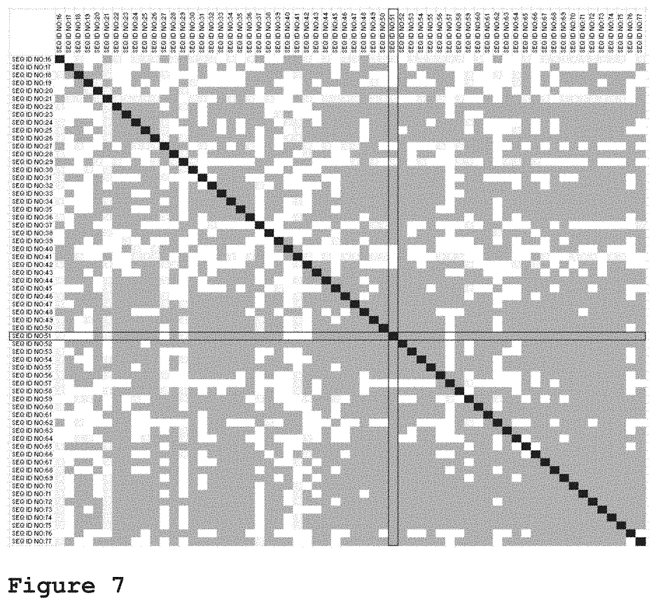

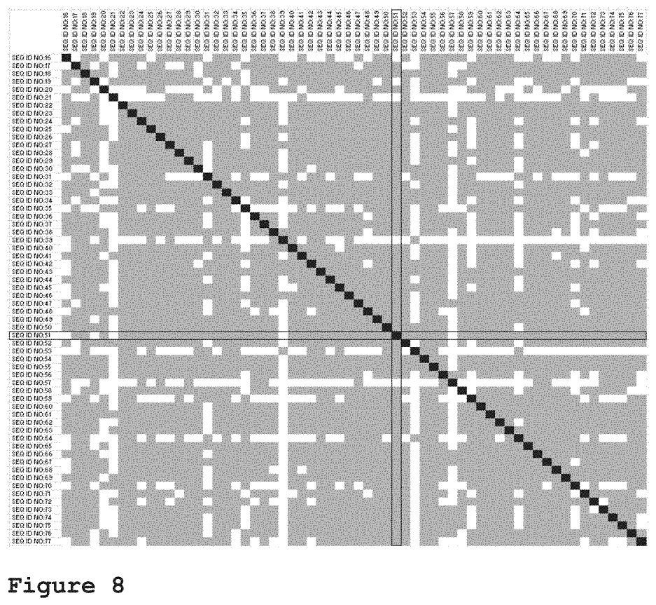

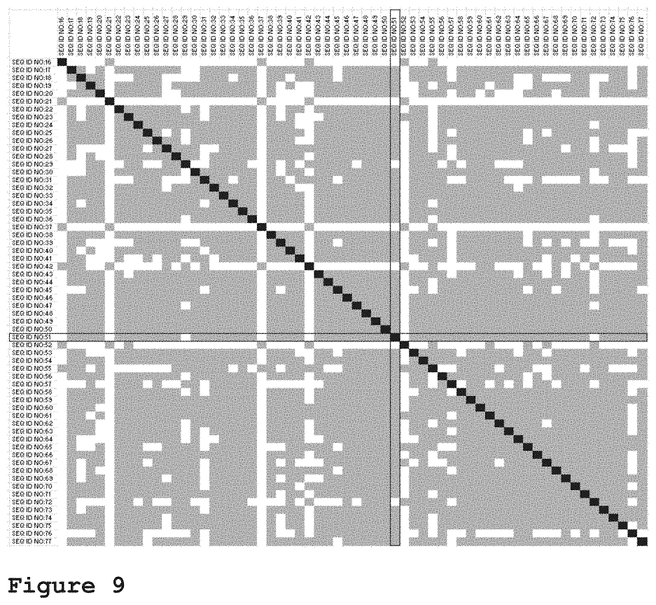

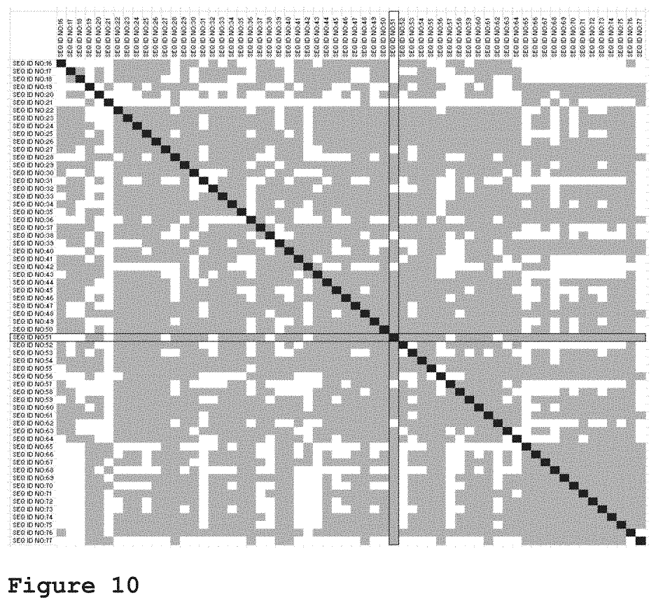

FIGS. 3 to 10 show the extent of co-methylation of the analyzed gene loci in the different types of malignant diseases. The gene loci are identified vertically and horizontally, each consecutively by SEQ ID NO: 16 (vertical: top, horizontal: left) to SEQ ID NO:77 (vertical: bottom, horizontal: right). SEQ ID NO:51 (black bordered) comprises SEQ ID NO: 13, which was examined in example 1 by quantitative real-time PCR to detect the DNA methylation of the CTLA4 gene.

The matrices show whether the DNA methylation of a specific sequence under investigation is correlated with the DNA methylation of the other sequences. A statistically significant correlation between the DNA methylation of two sequences, i. e. a p-value of the Spearman rank correlation of less than 0.05, is shown in the matrix as a grey box. White boxes indicate that there is no significant correlation (p≥0.05) of DNA methylation of the two corresponding sequences.

The results clearly show that all analyzed gene regions of CD28, CD80, CD86 and ICOS exhibited a DNA methylation that was significantly positively correlated with the DNA methylation of CTLA4 in the malignant diseases studied. Based on this co-methylation, the inventor was able to prove that, analogous to CTLA4, there is also an association between the DNA methylation of the genes CD28, CD80, CD86 and ICOS and the response of the malignant disease to the immunotherapy according to example 1. Consequently, as an alternative or in addition to the DNA methylation analysis of CTLA4, a DNA methylation analysis of one or more of the genes CD28, CD80, CD86 and ICOS enables the prediction of the response of a malignant disease to an immunotherapy designed to inhibit a PD-1 immune checkpoint signaling pathway.

From the co-methylation described above follows the further particular advantage of the invention that the DNA methylation analyses of the genes CTLA4, CD28, CD80, CD86 and ICOS can also functionally influence each other. Since, as shown above, for example a low DNA methylation of one of the genes of the present invention correlates with a low DNA methylation of another gene of the present invention, a combined DNA methylation analysis of both genes has the effect that both individual results complement each other to provide a particularly robust overall result with regard to the prediction of the response of the malignant disease to the immunotherapy. This mutual “consolidation effect” of the DNA methylation analysis of the genes of the present invention in predicting the response behavior is particularly advantageous for small sample quantities, where the amount of available DNA of a single immunoregulatory gene is close to the lower detection limit of a DNA methylation analysis. In this way, the present invention solves the problem that in routine clinical practice there are often only small tissue biopsies or liquid biopsies available with small amounts of DNA from cells of the malignant disease and/or immune cells, from which the response behavior of the malignant disease must be reliably predicted.

1: A method for predicting a response of a malignant disease to an immunotherapy designed to inhibit a Programmed cell death protein 1 (PD-1) immune checkpoint signaling pathway, comprising:
performing a DNA methylation analysis of at least a part of an immunoregulatory gene selected from the group consisting of CTLA4, CD86, CD28, CD80, ICOS, and any combination thereof of cells of the malignant disease and/or of immune cells interacting with the cells of the malignant disease, and
predicting the response of the malignant disease to the immunotherapy based on a presence, absence and/or level of DNA methylation of the immunoregulatory gene.

2: The method according to claim 1, wherein the DNA methylation analysis is performed on at least a part of each of at least two of the immunoregulatory genes.

3: The method according to claim 1, wherein the immunotherapy comprises a pharmaceutical compound which inhibits the PD-1 immune checkpoint signaling pathway by a binding of the pharmaceutical compound to PD-1, Programmed death ligand 1 (PD-L1), and/or Programmed death ligand 2 (PD-L2).

4: The method according to claim 1, wherein the immune cells interacting with the cells of the malignant disease is selected from the group consisting of T lymphocytes, B lymphocytes, antigen-presenting cells, natural killer cells, and any combination thereof.

5: The method according to claim 1, wherein the malignant disease is a disease selected from the group consisting of a melanoma, a carcinoma, a leukemia, a glioblastoma, a sarcoma, a lymphoma, and any combination thereof.

6: The method according to claim 1, wherein the response of the malignant disease to the immunotherapy is more responsive the lower the level of DNA methylation of the immunoregulatory gene.

7: The method according to claim 1, wherein the DNA methylation analysis comprises at least one gene copy of the immunoregulatory gene of a plurality of the cells of the malignant disease and/or of a plurality of the immune cells interacting with the cells of the malignant disease, and
wherein the response of the malignant disease to the immunotherapy is likely if less than or equal to 40% of the at least one gene copy of the immunoregulatory gene contains the DNA methylation.

8: A method of selecting a patient suffering from a malignant disease for an immunotherapy designed to inhibit a PD-1 immune checkpoint signaling pathway, comprising:
A) providing cells of the malignant disease and/or immune cells interacting with cells of the malignant disease from the patient,
B) performing a DNA methylation analysis of at least a part of an immunoregulatory gene selected from the group consisting of CTLA4, CD86, CD28, CD80, ICOS, and any combination thereof of the cells of the malignant disease and/or of the immune cells interacting with the cells of the malignant disease from step A),
C) selecting the patient for the immunotherapy based on a presence, absence, and/or level of DNA methylation of the immunoregulatory gene determined in step B).

9: A method, comprising:
performing a DNA methylation analysis of at least a part of an immunoregulatory gene selected from the group consisting of CTLA4, CD86, CD28, CD80, ICOS, and any combination thereof of cells of a malignant disease and/or of immune cells interacting with the cells of the malignant disease, and
a) predicting a response of the malignant disease to an immunotherapy;
b) individually selecting immunotherapy for the malignant disease; and/or
c) selecting a patient suffering from the malignant disease for the immunotherapy,
wherein the immunotherapy is designed to inhibit a PD-1 immune checkpoint signaling pathway.

10: A method, comprising:
designating a biomarker, wherein the biomarker comprises a presence, absence or level of DNA methylation of at least a part of an immunoregulatory gene selected from the group consisting of CTLA4, CD86, CD28, CD80, ICOS, and any combination thereof of cells of a malignant disease and/or of immune cells interacting with the cells of the malignant disease, and
i) predicting a response of the malignant disease to an immunotherapy;
ii) individually selecting an immunotherapy for the malignant disease; and/or
iii) selecting a patient suffering from the malignant disease for an immunotherapy,
wherein the immunotherapy is designed to inhibit the PD-1 immune checkpoint signaling pathway.

11: The method according to claim 9, wherein the immunotherapy is further designed to inhibit a CTLA4 immune checkpoint signaling pathway.

12: A kit for carrying out the method according to claim 1, comprising
a reagent for the DNA methylation analysis of at least the part of the immunoregulatory gene selected from the group consisting of CTLA4, CD86, CD28, CD80, ICOS, and any combination thereof of the cells of the malignant disease and/or of the immune cells interacting with the cells of the malignant disease to determine the presence, absence, and/or level of DNA methylation of the immunoregulatory gene.

13: The kit according to claim 12, wherein the DNA methylation analysis is performed on at least a part of each of at least two of the immunoregulatory genes.

14: The method according to claim 1, wherein the immunotherapy comprises an anti-PD-1 antibody, an anti-PD-L antibody, and/or an anti-PD-L2 antibody.

15: The method according to claim 5, wherein the malignant disease comprises the carcinoma, and wherein the carcinoma is a squamous cell carcinoma or an adenocarcinoma.

16: The method according to claim 1, wherein the absence of DNA methylation of the immunoregulatory gene indicates that the malignant disease is likely to respond to the immunotherapy.

17: The method according to claim 11, wherein the immunotherapy comprises an antibody selected from the group consisting of an anti-CTLA4 antibody, an anti-CD80 antibody, an anti-CD86 antibody, an anti-CD28 antibody, and any combination thereof.

18: A method for immunotherapeutic treatment of a patient suffering from a malignant disease with a pharmaceutical compound designed to inhibit a PD-1 immune checkpoint signaling pathway, comprising:
I) performing a DNA methylation analysis of at least a part of an immunoregulatory gene selected from CTLA4, CD86, CD28, CD80, ICOS, and any combination thereof from cells of said malignant disease and/or immune cells interacting with the cells of said malignant disease prior to and/or during said immunotherapeutic treatment,
predicting a response of said malignant disease to said pharmaceutical compound based on a presence, absence and/or level of DNA methylation of said immunoregulatory gene, and
II) administering the pharmaceutical compound if the DNA methylation analysis indicates that said malignant disease is likely to respond to immunotherapeutic treatment with said pharmaceutical compound, or
III) not administering, discontinuing or reducing the administering of the pharmaceutical compound if step I) indicates that the malignant disease is unlikely to respond or is no longer likely to respond to the immunotherapeutic treatment with said pharmaceutical compound.

19: The method of claim 18, further comprising:
administering an additional pharmaceutical compound which is designed to inhibit a CTLA4 immune checkpoint signaling pathway.

20: The method of claim 19, wherein the additional pharmaceutical compound is selected from the group consisting of an anti-CTLA4 antibody, an anti-CD80 antibody, an anti-CD86 antibody, an anti-CD28 antibody, or any combination thereof.
